(19) United States
(12) Patent Application Publication
(10) Pub. No.: US 2025/0204775 A1
(43) Pub. Date: Jun. 26, 2025

(54) DEVICE AND METHOD FOR DETERMINING A SHIFT IN A REFRACTION VALUE OF AN EYE OF A SUBJECT INDUCED BY AN OPTICAL FILTER

(71) Applicant: Essilor International, Charenton-le-Pont (FR)

(72) Inventors: Marie DUBAIL, Saint-Maurice (FR); Anne-Catherine SCHERLEN, Ranspach (FR); Sébastien FRICKER, Creteil (FR)

(73) Assignee: Essilor International, Charenton-le-Pont (FR)

(21) Appl. No.: 18/852,150

(22) PCT Filed: Mar. 30, 2023
(86) PCT No.: PCT/EP2023/058358

§ 371 Date: Sep. 27, 2024

(30) Foreign Application Priority Data
Mar. 31, 2022 (EP) 22305423.0

(51) Int. Cl. A61B 3/103; A61B 3/00

(52) U.S. Cl. CPC A61B 3/103; A61B 3/0025

(57) ABSTRACT

A device for determining a shift in a refraction value of an eye lighted by a light beam emitted by a light source and transmitted through an optical filter, wherein the device includes a memory storing a value of an initial spectral feature of the light source, a value of an optical feature of the optical filter, and a refraction shift model linking a magnitude associated with the shift and a spectral feature of the light beam. Further, the device includes processors programmed to determine a value of a spectral feature of the light beam based on the value of the initial spectral feature of the light source and the value of the optical feature of said optical filter; and determine the shift based on the refraction shift model and the value of the spectral feature of the light beam.

### TECHNICAL FIELD OF THE DISCLOSURE

The disclosure relates to a device and method for determining a shift in a refraction value of an eye of a subject induced by an optical filter.

### BACKGROUND INFORMATION AND PRIOR ART

When a light beam is transmitted to an eye of a subject, different wavelengths of visible light are focused on different distances of the retina where the image seen by the subject is formed.

Light with long wavelengths, corresponding for example to red light, is focused on points located slightly behind the retina, while light with short wavelengths, for example blue light, is focused slightly in front of the retina. The amplitude of this defocus, i.e., the distance between the point of focus and the retina depends on the wavelength and is called “Longitudinal chromatic aberration” (LCA).

This phenomenon implies that images of different colors, i.e. of different wavelengths, or components of an image corresponding to different colors or different wavelengths may have different sharpness when seen by the eye of the subject.

Meanwhile the use of colored lenses is more and more widespread, and the colored lenses are more and more specific and complex. Colored lenses are polychromatic optical filters.

Optical filters used in eyeglasses aim to protect the ocular system and to improve comfort and visual performance in different light conditions. Colorful filters are also proposed for aesthetic purposes. They may be associated with lenses having a corrective power or not.

Because of the different focus locations of light beams having different wavelengths in the eye, the use of an optical filter that changes the wavelengths distribution of the light transmitted to the eye of the subject may modify the optimal point of focus on the retina.

Experimental results from the Applicant show that a high proportion of long wavelengths versus short wavelengths in the light reaching the eye induces a significant hyperopic shift. A high proportion of short wavelengths versus long wavelengths results in a significant myopic shift. The amplitude of the myopic shift is higher than the amplitude of the hyperopic shift.

The ametropic shift resulting from the presence of an optical filter in front of the eyes of a person can induce fatigue and reduce visual performances such as visual acuity, contrast sensitivity, reading speed.

In other words, adding an optical filter in front of the eye may induce an ametropic shift of the visual perception of the subject and may decrease the comfort and the quality of vision of the subject.

In particular, when the refraction features of the eye are determined in order to deduce the corrective power of a corrective ophthalmic lens to improve the vision of the subject, the refraction features of the eye are determined in order to focus on the retina light with wavelength comprised between green and red light, in order for the defocus associated with red light to be equivalent to the defocus associated with green light.

The refraction feature of the eye, and consequently the corrective power of the corrective ophthalmic lens, is determined with ambient natural or artificial white light, for clear lenses having negligible absorption of visible light.

The addition of an optical filter may then alter the corrective effect on the eye of the subject of the corrective ophthalmic lens thus determined.

In order to solve this problem, one could consider measuring the refractive error of the wearer with the actual spectral transmission of the chosen lenses or optical filters to allow a perfect focus of the light on the retina and produce an adapted prescription. The wearer will have a sharper vision and produce less accommodative effort.

Yet, precise methodology and precise instrument are required to get such accurate measurements and these measurements would be long and tedious.

### SUMMARY OF THE INVENTION

Therefore, one object of the invention is to provide a device for determining quickly and easily the shift in the refraction value of an eye introduced by an optical filter.

The above object is achieved according to the invention by providing a device for determining a shift in a refraction value of an eye of a subject lighted by a light beam emitted by a light source, said shift being induced by an optical filter through which said light beam is transmitted to the eye of the subject, said device comprising one or more memories and one or more processors, wherein:
said one or more memories have in memory:
one or more values of one or more initial spectral features of said light source,
one or more values of one or more optical features of said optical filter, and
a refraction shift model linking a magnitude associated with said shift and one or more spectral features of the light beam transmitted to the eye of the subject; and wherein
said one or more processors are programmed to perform the following steps:
determining one or more values of one or more spectral features of said light beam transmitted to the eye of the subject through said optical filter based on said one or more values of one or more initial spectral features of said light source and said one or more values of one or more optical features of said optical filter; and
determining said shift in the refraction of the eye of the subject based on said refraction shift model and said values of the spectral features of said light beam transmitted to the eye of the subject through the optical filter.

Thanks to the device according to the invention, the shift in refraction of the eye of the subject induced by an optical filter may be determined with minimal measurements, based on optical features of the optical filter that may be predetermined, known by any kind of means or measured.

Thanks to the determination of the ametropic shift with the device according to the invention, it is then possible to consider either:
adapting the prescription based on the optical features of the optical filter to improve the quality of vision or visual comfort of the subject, for example by compensating the refraction shift induced by the filter with the power of the corrective lenses,
using the refraction shift induced by the optical filter to improve the visual performance of the subject in specific conditions. For example, it is possible to consider using a myopic shift in near vision tasks to play the role and partly replace a spherical power of a corrective lens.

Spherical power of the ophthalmic corrective lens may be replaced by a color of this lens for specific activities of the subject.

The device according to the invention provides a tool to predict the defocus associated with an optical filter having any spectral transmission, without making any actual measurement on the subject.

Taking into account said defocus allows taking into account the corresponding ametropic shift, therefore providing a better visual performance and reducing fatigue thanks to an adapted prescription of the corrective ophthalmic lens taking into account the optical filter.

Other advantageous features of the device according to the invention are the following:
said one or more values of one or more initial spectral features of said light source comprise an initial spectrum of said light emitted by said light source and said one or more values of one or more optical features of said optical filter comprise values quantifying the transmission of light through said optical filter;
said one or more processors are programmed to determine a spectrum of said light beam transmitted to the eye of the subject through said optical filter for determining said one or more values of one or more spectral features of said light beam transmitted to the eye of the subject though said optical filter;
said one or more memories further have in memory data relative to one or more optical features of the eye of the subject and said one or more processors are programmed to take into account these data for determining one or more values of one or more spectral features of said light beam transmitted to the eye of the subject through said optical filter;
said data relative to one or more optical features of the eye comprise data relative to the transmission of light through a front part of the eye to the retina of the eye;
said one or more memories have in memory weighting factors representative of a light sensitivity of the retina of the eye of the subject at one or more wavelengths and wherein said one or more processors are programmed to determine said one or more values of one or more spectral features of said light beam transmitted to the eye of the subject through said optical filter taking into account these weighting factors;
said one or more spectral features of the light beam transmitted to the eye of the subject through said optical filter comprise a statistical value of wavelength representative of the wavelength distribution of a spectrum of said light beam transmitted to the eye of the subject through said optical filter;
said statistical value of wavelength representative of the wavelength distribution of said spectrum comprises a wavelength barycenter of said spectrum;
said one or more memories have in memory data relative to a longitudinal chromatic aberration of the eye depending on a wavelength of a light beam transmitted to the eye and wherein said refraction shift model comprises a relationship between the shift in refraction of the eye of the subject and the longitudinal chromatic aberration of the eye of the subject at said statistical value of wavelength;
said one or more values of one or more initial spectral features of said light source comprise an initial spectrum of said light emitted by said light source;
said one or more processors are programmed to determine a reference statistical value of wavelength representative of the wavelength distribution of a reference spectrum of a reference light beam emitted by the light source and transmitted to the eye of the subject in the absence of the optical filter based on said initial spectrum of said light emitted by said light source; and said refraction shift model takes into account the longitudinal chromatic aberration of the eye of the subject at said reference statistical value of wavelength;
said refraction shift model provides a shift value equal to the difference between longitudinal chromatic aberration of the eye at the statistical value of wavelength and the longitudinal chromatic aberration of the eye at said reference statistical value of wavelength;
said one or more spectral features of the light beam transmitted to the eye of the subject through the optical filter comprise a spectrum of said light beam transmitted to the eye through said optical filter on a predetermined global range of wavelengths;
said one or more memories have in memory elementary shift values associated with predetermined elementary range of wavelengths, and said refraction shift model comprises a relationship between the refraction shift and a sum, for all of said elementary range of wavelengths comprised in said predetermined global range of wavelengths, of said elementary shift values weighted by an integral of the spectrum of said light transmitted to the eye through said filter within the corresponding elementary range of wavelengths;
said refraction shift model provides a shift value equal to a ratio between said sum and an integral of said spectrum of said light transmitted to the eye through said filter within the predetermined global range of wavelengths;
said elementary shift values are determined taking into account a database comprising a plurality of test spectra of light transmitted to an eye and the corresponding measured values of refraction shift in said eye, by minimizing the difference between each measured value of refraction shift and a calculated value of refraction shift, said calculated value of the refraction shift being determined using said relationship and the test spectrum of light transmitted to the eye to which said measured value of refraction shift corresponds.

The invention also relates to an adaptive eyewear for a subject comprising:
ophthalmic lenses having variable power and/or a variable filter whose variations are controlled by a controller,
a device as described above,
wherein said device is in communication with said controller and said controller is programmed to determine a power variation and/or a tint variation of said ophthalmic lenses taking into consideration the shift in the refraction of the eye of the subject determined by said device.

The invention also relates to a method for determining a shift in a refraction value of an eye of a subject lighted by a light beam emitted by a light source, said shift being induced by an optical filter through which said light beam is transmitted to the eye of the subject, said device comprising one or more memories and one or more processors, comprising the following steps:
determining one or more values of one or more initial spectral features of said light source,
determining one or more values of one or more optical features of said optical filter, and
providing a refraction shift model linking a magnitude associated with said shift and one or more spectral features of the light beam transmitted to the eye of the subject;
determining one or more values of one or more spectral features of said light beam transmitted to the eye of the subject through said optical filter based on said one or more values of one or more initial spectral features of said light source and said one or more values of one or more optical features of said optical filter; and
determining said shift in the refraction of the eye of the subject based on said refraction shift model and said values of the spectral features of said light beam transmitted to the eye of the subject through the optical filter.

### DETAILED DESCRIPTION OF EXAMPLE(S)

The following description with reference to the accompanying drawings will make it clear what the invention consists of and how it can be achieved. The invention is not limited to the embodiments illustrated in the drawings. Accordingly, it should be understood that where features mentioned in the claims are followed by reference signs, such signs are included solely for the purpose of enhancing the intelligibility of the claims and are in no way limiting on the scope of the claims.

In the accompanying drawings:

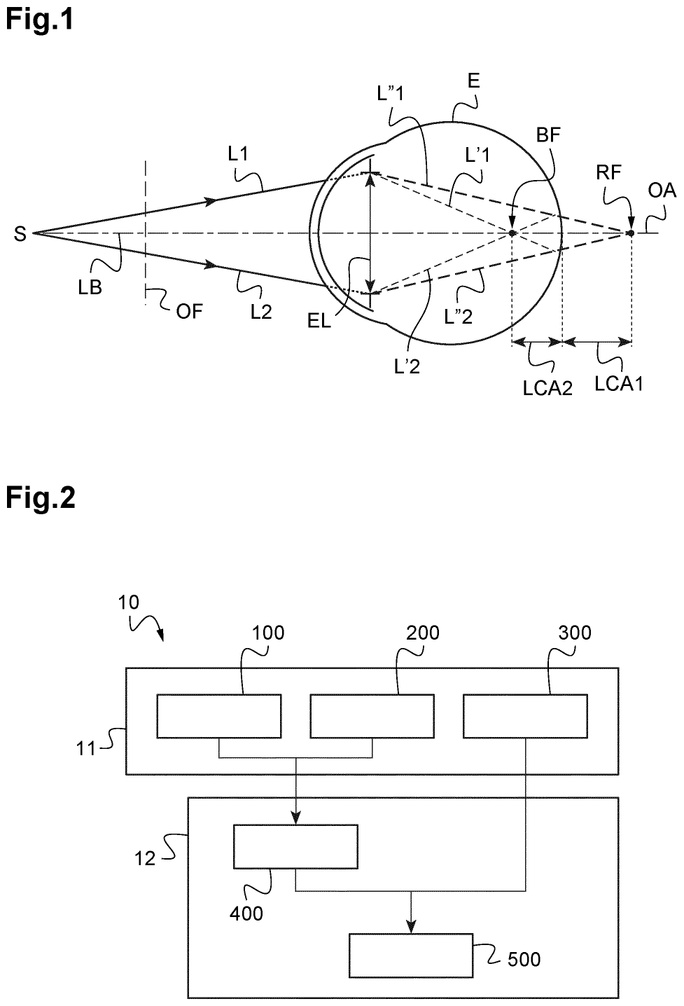

FIG. 1 is a schematic view of a section of an eye showing examples of longitudinal chromatic aberration,

FIG. 2 is a block diagram showing the main parts of the device according to the invention and the steps of the method performed by this device,

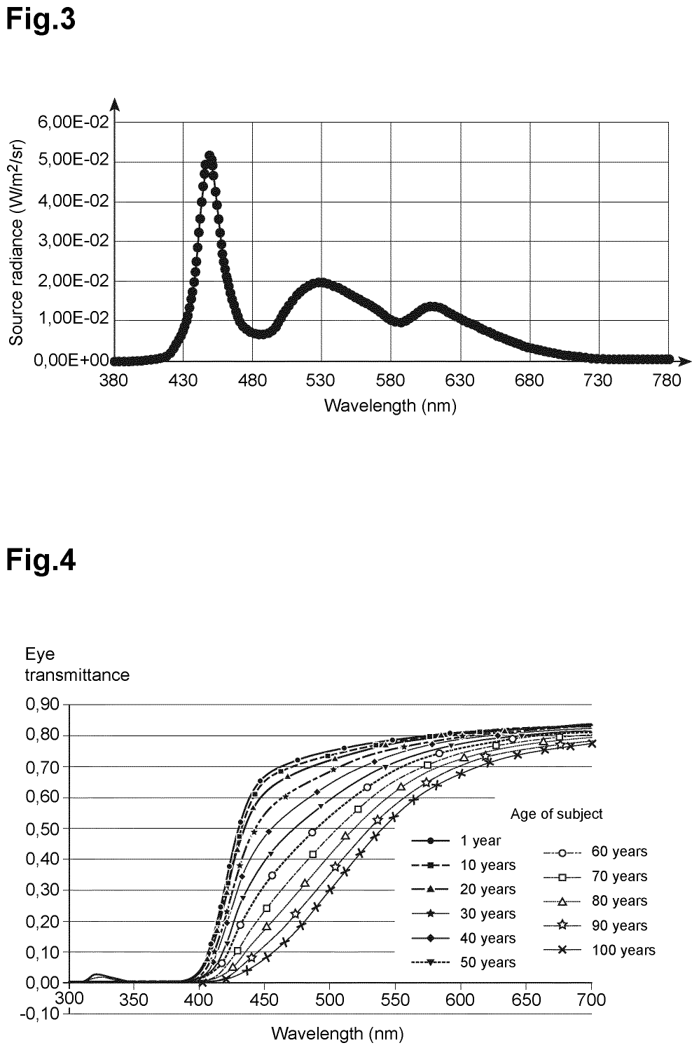

FIG. 3 is an example graph of the radiance of a commercial light source plotted against the wavelength,

FIG. 4 is a graphical representation of the total transmittance of the clear ocular media of the human eye at different ages plotted against the wavelength,

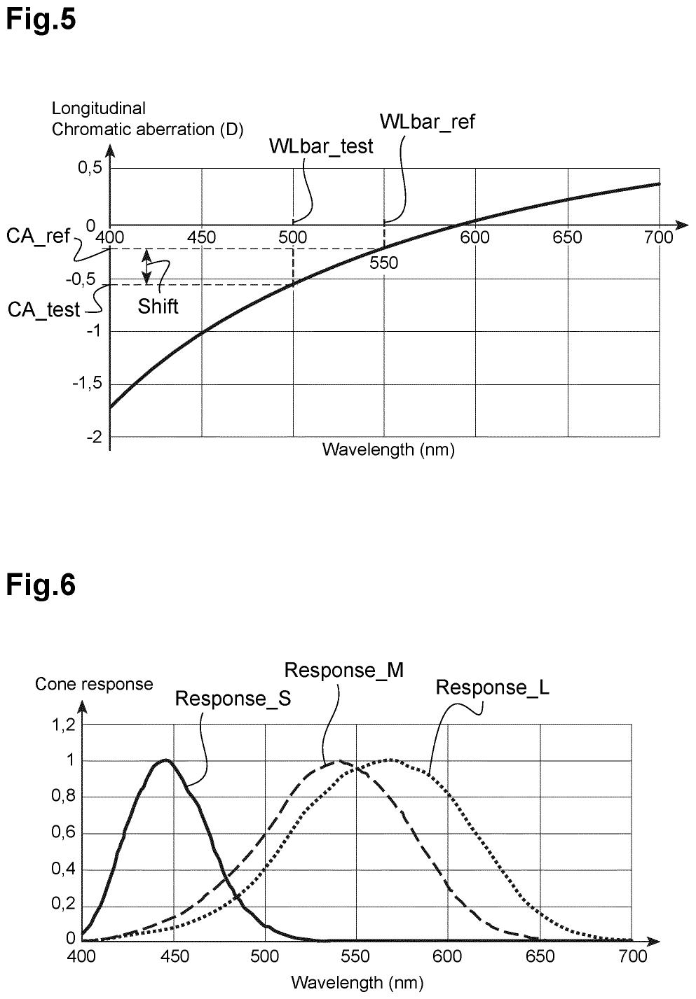

FIG. 5 is a graphical representation of the longitudinal chromatic aberration of an eye as a function of wavelength,

FIG. 6 is a graphical representation of the response curves of the three different types of cones of the human eye as a function of wavelength, the cone response being the relative spectral sensitivity of L, M and S cones and being correlated to the probability of absorption of photons reaching the retina after filtration by the crystalline lens and macular pigment or the front part of the eye, also called anterior ocular media (no specific unit),

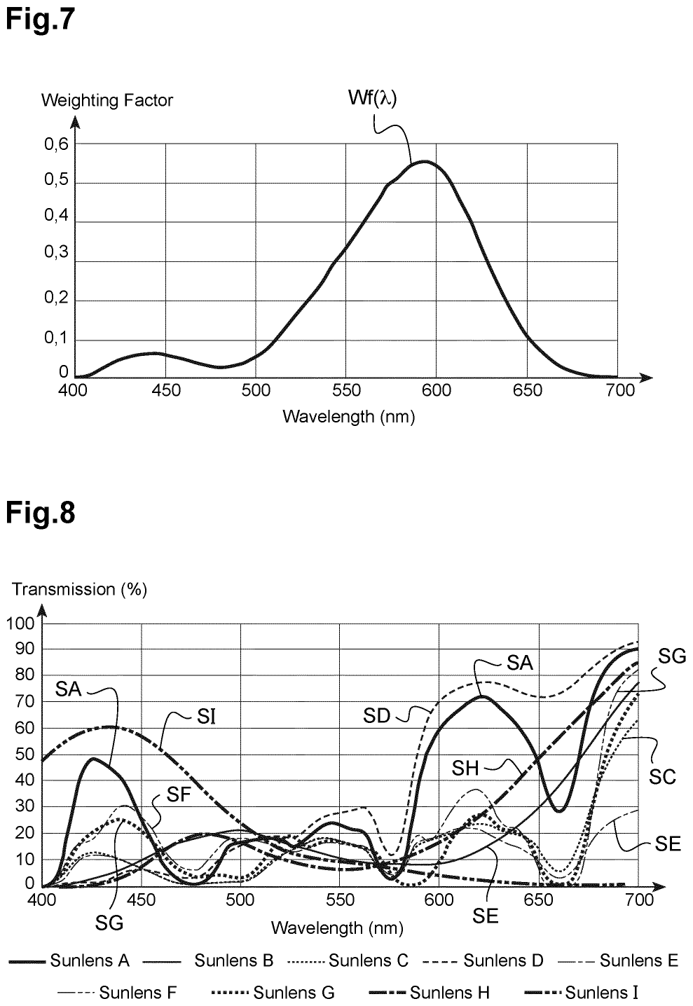

FIG. 7 is a graphical representation of a weighing factor of cones response as a function of wavelength,

FIG. 8 is a graphical representation of different filters transmission spectra as a function of wavelength,

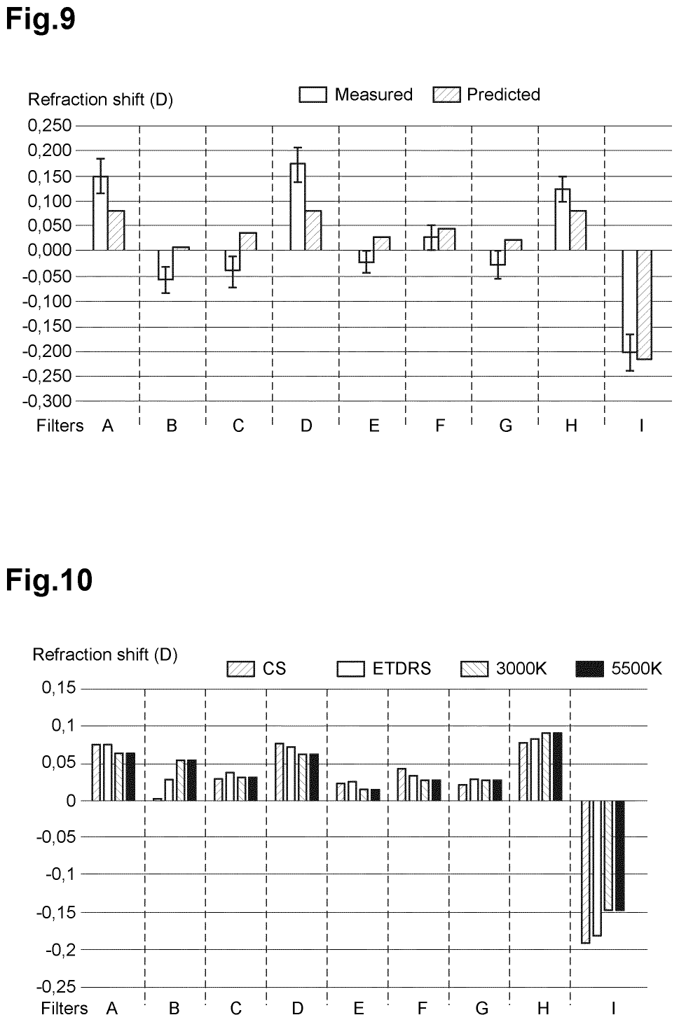

FIG. 9 is a graphical representation of the ametropic shift measured and calculated by a first embodiment of the device of the invention for different filters,

FIG. 10 is a graphical representation of the ametropic shift calculated by the first embodiment device of the invention for different filters and different light sources,

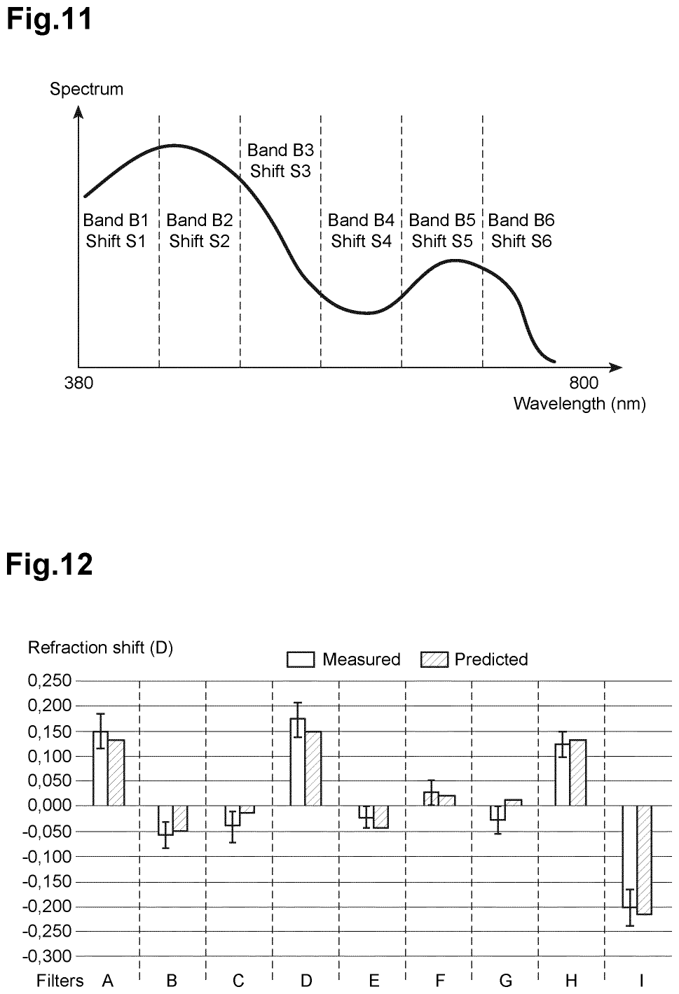

FIG. 11 is a graphical representation of the wavelength ranges considered for an example of a light source spectrum in a second embodiment of the device of the invention,

FIG. 12 is a graphical representation of the ametropic shift measured and calculated by the second embodiment of the device of the invention for different filters.

As mentioned in the introductive part and illustrated on FIG. 1, because of the Longitudinal Chromatic Aberration of the human eye E, light beams having different wavelengths exhibit different focus locations BF, RF of in the eye E.

FIG. 1 shows the path of a light beam LB delimited by two rays of light L1, L2 incident on the eye E. The eye lens EL focuses the part of said light beam LB having shorter wavelengths, corresponding to blue colors, in front of the retina at point BF corresponding to rays of light L′1, L′2, whereas it focuses the part of said light beam LB having longer wavelengths, corresponding to red colors, behind the retina of the eye at point RF corresponding to rays of light L″1, L″2. The defocus of the red light beam is the distance LCA1 between the retina and point RF. The defocus of the blue light beam is the distance LCA2 between the retina and point BF.

The situation considered in the following is schematically shown on FIG. 1: the eye E of the subject receives said light beam LB emitted by a light source S having given spectral features. An optical filter OF, shown in dashed line on FIG. 1, is placed on the path of the light beam LB, between the light source S and the eye E.

Such an optical filter OF can comprise a tinted optical element such as a tinted substrate, or can comprise a tinted optical film associated with a base substrate that is clear or also tinted. The tint of the film/substrate can be obtained from a dye composition comprising, at least, one absorptive dye or a combination of different absorptive dyes, in order to define a colored film/substrate, a sunglass film/substrate, a filtering film/substrate filtering specific wavelength ranges of the light source such as anti-UV or blue-cut film/substrate, or a polar film/substrate.

Generally speaking, the tinted film/substrate is defined so as to reduce the visible light transmission Tv of illuminant D65 to respect specific categories of sunglasses:
an optical element having 80% or more visible light transmission Tv, also referred to as a “category 0” optical element,
an optical element having between 46% and 79% visible light transmission Tv, also referred to as a “category 1” optical element,
an optical element having between 18% and 45% visible light transmission Tv, also referred to as a “category 2” optical element.

The above-described optical filter may also comprise at least one absorptive dye that is activable. The various types of activable dyes are well known to the person of ordinary skill in the art. As a result, the optical filter may be switchable between different configurations i.e. switchable between an active and an inactive states upon an external source such as an external light source. Activable dyes comprise for example photochromic or electrochromic type dyes.

The use of the optical filter OF changes the wavelengths distribution of the light beam LB transmitted to the eye E of the subject and may then globally modify the sharpness of the image seen by the subject.

The invention provides a device 10 and a methodology to predict the defocus induced by the optical filter OF for the eye E of the subject, without having to perform any measurement involving said subject.

More precisely, said device is a device 10 for determining a shift in a refraction value of the eye E of the subject lighted by the light beam LB emitted by the light source S, said shift being induced by the optical filter OF through which said light beam LB is transmitted to the eye E of the subject. This device 10 is schematically represented on FIG. 2. Said device 10 comprises one or more memories 11 and one or more processors 12, wherein:
said one or more memories 11 have in memory:
one or more values 100 of one or more initial spectral features of said light source S,
one or more values 200 of one or more optical features of said optical filter OF, and
a refraction shift model 300 linking a magnitude associated with said shift and one or more spectral features of the light beam LB transmitted to the eye E of the subject; and wherein
said one or more processors 12 are programmed to perform the following steps:
a) determining 400 one or more values of one or more spectral features of said light beam LB transmitted to the eye E of the subject through said optical filter OF based on said one or more values 100 of one or more initial spectral features of said light source S and said one or more values 200 of one or more optical features of said optical filter OF; and
b) determining 500 said shift in the refraction of the eye E of the subject based on said refraction shift model 300 and said values of the spectral features of said light beam LB transmitted to the eye E of the subject through the optical filter OF.

Said one or more values 100 of one or more initial spectral features of said light source S comprise for example an initial spectrum of said light emitted by said light source S.

Said one or more values 200 of one or more optical features of said optical filter OF comprise for example values quantifying the transmission of light through said optical filter OF.

An example of the initial spectrum of said light source is shown on FIG. 3. This figure shows a graph of the radiance of a commercial screen as a function of wavelength. Alternatively, the initial spectrum of the light source may comprise a graph of the intensity, luminosity, luminance or any linked magnitude as a function of wavelength. The initial spectrum of the light source may also be memorized as a standard illuminant, a value of luminance or luminosity, a table of values of radiance, intensity, or other linked magnitude as a function of wavelength.

This initial spectrum may be measured thanks to a spectrometer or may be retrieved from the built-in information of said source.

Said one or more optical features of said optical filter OF may comprise a graph or a table of values of the transmission or transmittance of light through said optical filter OF.

The optical features of said optical filter OF may also comprise an opto-geometrical description of the film and/or substrate constitutive of the optical filter OF, including the geometry of the diopters, their relative positionings, and the refractive index of the material(s).

The optical features of said optical filter OF may also comprise information about the longitudinal chromatic aberration generated by the filter itself.

The value of transmittance of the filter at a given wavelength is equal to the luminance of the light beam transmitted by the filter divided by the luminance of the incident light beam at said wavelength. The value of transmittance of the filter at a given wavelength may also be defined as the radiance of the light beam transmitted by the filter divided by the radiance of the incident light beam at said wavelength.

The value of transmission of the filter at a given wavelength is equal to the intensity of the light beam transmitted by the filter divided by the intensity of the incident light beam at said wavelength.

The information about the longitudinal chromatic aberration can be provided as the material dispersion, or the value of the longitudinal chromatic aberration, or the coefficient of longitudinal chromatic aberration.

Said one or more values of one or more spectral features of said light beam LB transmitted to the eye E of the subject through said optical filter OF may comprise a spectrum of said light beam transmitted through the filter determined by multiplying the initial spectrum of the light source by the values quantifying the transmission of light through said optical filter OF at each wavelength.

The spectral features of the light beam transmitted to the eye may also comprise the amount of longitudinal chromatic aberration within the beam of light, for example within the range of the wavelengths that are included in the light beam.

In this case, said one or more processors 12 are programmed to determine in step a) a spectrum of said light beam LB transmitted to the eye E of the subject through said optical filter OF.

For example, the initial spectrum of the light source comprises intensity, luminance or radiance values for a set of wavelengths values and the values of one or more optical features of said optical filter OF comprise transmission or transmittance values of the filter for said set of wavelengths. The values of the spectral features of the light beam LB transmitted to the eye comprise the values of the intensity of the light source multiplied by the transmission of the filter for each wavelength of said set or the values of the luminance or the radiance of the light source multiplied by the transmittance of the filter for each wavelength of said set.

Naturally, other factors or parameters may be taken into account.

Optionally, said one or more memories 11 may further have in memory data relative to one or more optical features of the eye E of the subject and said one or more processors 12 are programmed to take into account these data for determining said one or more values of one or more spectral features of said light beam LB transmitted to the eye E of the subject through said optical filter OF.

Said data relative to one or more optical features of the eye E may for example comprise data relative to the transmission of light through a front part of the eye E to the retina of the eye E. The front part of the eye E corresponds to the part of the eye E located in front of the retina. This front part of the eye E indeed presents predetermined transmission features and acts as an additional optical filter placed in front of the retina, where the cells detecting the light beam are located.

In this case, said one or more processors 12 are programmed to determine in step a) a spectrum of said light beam LB transmitted to the eye E of the subject through said optical filter OF for example by multiplying the values of the intensity or luminance of the light source by the transmission or the transmittance of the filter for each wavelength of said set and by the transmission or transmittance of the front part of the eye for each wavelength.

The transmission or transmittance of the optical filter OF and/or of the front part of the eye E may be measured in a preliminary calibration step or may be retrieved from a database.

The transmission or transmittance of the front part of the eye E may comprise specific values customized for the subject, either by measures on the subject or by taking into account personal features of the subject such as the age of the subject or spectral transmission of the front part of the eye or anterior ocular media.

Examples of data relative to the total transmittance of the front part of the eye depending on wavelength for subject of different ages are presented on FIG. 4. The data is shown here as a graph of transmittance of the front part of the eye versus wavelength.

Alternatively, the transmission or transmittance of the front part of the eye E may comprise average transmission or transmittance values determined or a reference population of subjects or for any subject.

In a more refined embodiment, said one or more memories 11 have in memory weighting factors representative of a light sensitivity of the retina of the eye E of the subject at one or more wavelengths. Said one or more processors 12 are then programmed to determine said one or more values of one or more spectral features of said light beam LB transmitted to the eye E of the subject through said optical filter OF taking into account these weighting factors.

Such a weighting factor may be derived based on response curves Response_S, Response_M, Response_L of the retinal cells detecting the light beam, for example on the basis of three types of cones noted in the following S, M and L. Data relative to the response curves of the cone of the eye are available from the CIE (Commission Internationale de l'Eclairage) and is represented on FIG. 6 showing the light sensitivity of each type of cone depending on wavelength. Light sensitivity may be defined as the function representing the number of photons absorbed by the retina for each wavelength.

The weighting factor Wf taken into account may be determined as a weighted average of the response of the three types of cones for each wavelength. It is for example calculated as Wf(λ)=ws*Response_S(λ)+wl*Response_L(λ)+wm*Response_M(λ), where ws, wm, and wl are three scalars determining the weight of each cone response. An example showing how these scalars are determined will be developed later.

The refraction shift model 300 linking a magnitude associated with said shift and one or more spectral features of the light beam LB transmitted to the eye E of the subject may be determined by using machine learning algorithms trained on a reference database.

A preliminary step of the method according to the invention may therefore comprise building the reference database comprising measured refractive error values of reference subjects.

This reference database is for example built based on the results of a psychovisual study on a group of reference subjects. The refractive errors measured on these reference subjects presented with different optical filters with predetermined known spectral features are used to model the ametropic shift induced by a predetermined optical filter.

In the examples described below, the reference database comprises data gathered by a psychovisual study on 30 young adults. Measurements of ametropic shifts have been conducted. For each combination of a subject and an optical filter, monocular equivalent sphere (MES) was measured for each eye by a subjective refraction method. The same measurement was performed without the optical filter. The age of each subject is also known.

Two different embodiments are described in the following.

The approach used in the first embodiment uses the physiological parameters of the eye to calculate the defocus.

The approach used in the second embodiment is based on experimental data to determine a defocusing function.

### First Embodiment

According to this embodiment, said one or more values of one or more initial spectral features of said light source comprise an initial spectrum of said light emitted by said light source. The one or more processors 12 are programmed to determine one or more values of one or more reference spectral features of a reference light beam emitted by said light source and reaching the retina of the eye E of the subject in the absence of any added optical filter OF.

For example, the one or more processors 12 are programmed to determine a reference spectrum Spect_ref of the reference light beam. This reference spectrum Spect_ref is for example determined by multiplying the initial spectrum of the light source Spect_source by the transmission or transmittance of the front part of the eye Trans_eye, for each wavelength, or:

$$Spect\_ref ( λ ) = Spect\_source {( λ )}^{*} Trans\_eye ( λ ) .$$

The one or more processors are also programmed to determine one or more test spectral features of a test light beam emitted by said light source and reaching the retina of the eye of the subject through an added optical filter OF.

For example, the one or more processors 12 are programmed to determine a test spectrum of the test light beam. This test spectrum Spect_test is for example determined by multiplying the reference spectrum Spect_ref of the light source by the transmission or transmittance of the optical filter Trans_filter at each wavelength,

$$Spect\_test ( λ ) = Spect\_ref {( λ )}^{*} Trans\_filter ( λ ) .$$

Said one or more spectral features of the light beam transmitted to the eye of the subject through said optical filter, i.e. the test light beam, may comprise a statistical value of wavelength representative of the wavelength distribution of the spectrum of said light beam transmitted to the eye of the subject through said optical filter, i.e. the test spectrum. In the following, this value will be named “test statistical value of wavelength”.

Similarly, said one or more spectral features of the light beam transmitted to the eye E of the subject in the absence of said optical filter OF, i.e. the reference light beam, may comprise a statistical value of wavelength representative of the wavelength distribution of the reference spectrum of the reference light beam emitted by the light source and transmitted to the eye of the subject in the absence of the optical filter.

Said one or more processors 12 are programmed to determine said statistical value of wavelength representative of the wavelength distribution of the reference spectrum. In the following, this value will be named “reference statistical value of wavelength”. The reference statistical value of wavelength is determined based on said initial spectrum of said light emitted by said light source.

For example, said statistical value of wavelength representative of the wavelength distribution of said test spectrum, respectively of said reference spectrum, comprises a wavelength barycenter WLbar_test, WLbar_ref of said test spectrum, respectively of said reference spectrum.

The wavelength barycenter WLbar_test, WLbar_ref may be determined as the weighted mean of the wavelengths of the spectrum, with weight factors equal to the normalized value of the spectrum at each wavelength (in intensity, luminance, radiance, luminosity . . . ). The following formula may be used to determine said wavelength barycenter:

$$W L b a {r}_{t e s t} = \frac{{Σ}_{i} {λ}_{i} S p e c {t}_{t e s t} ( {λ}_{i} )}{{Σ}_{i} S p e c {t}_{t e s t} ( {λ}_{i} )}$$

$$WLba {r}_{r e f} = \frac{{Σ}_{i} {λ}_{i} S p e c {t}_{r e f} ( {λ}_{i} )}{{Σ}_{i} S p e c {t}_{r e f} ( {λ}_{i} )}$$

In this first embodiment, said one or more memories 11 have moreover in memory data relative to a longitudinal chromatic aberration of the eye depending on a wavelength of a light beam transmitted to the eye.

An example of such data is shown on FIG. 5 where a graph of the longitudinal chromatic aberration of an eye in diopter versus wavelength is represented.

Alternatively, said data may comprise a table of values of the longitudinal chromatic aberration associated with the corresponding wavelength.

The longitudinal chromatic aberration of the eye put in memory may be an average longitudinal chromatic aberration for the human eye or a customized longitudinal chromatic aberration. The customized longitudinal chromatic aberration may be determined based on personal features of the subject such as age, gender, type of ametropia. It can be determined based on statistical values of longitudinal chromatic aberration for a population of subjects. The longitudinal chromatic aberration of the eye of the subject may also be measured.

Said refraction shift model then comprises a relationship between the shift in refraction of the eye of the subject and the longitudinal chromatic aberration of the eye of the subject at said statistical value of wavelength.

In practice, said refraction shift model takes into account the longitudinal chromatic aberration of the eye of the subject at said reference statistical value of wavelength.

More precisely, said refraction shift model provides a shift value S equal to the difference between longitudinal chromatic aberration LCA(WLbar_test) of the eye at the test statistical value of wavelength WLbar_test and the longitudinal chromatic aberration LCA(WLbar_ref) of the eye at said reference statistical value of wavelength WLbar_ref (see FIG. 5):

$$S = CA\_test - CA\_ref with CA\_test = LCA ( WL\_test ) and CA\_ref = LCA ( WLbar\_ref ) .$$

Optionally, the refraction shift model can take into account the longitudinal chromatic aberration of the substrate of the optical filter, in addition to the longitudinal chromatic aberration of the eye. In that case, the longitudinal chromatic aberration values LCA(WLbar_test) or LCA(WLbar_ref) to consider are the sum of the longitudinal chromatic aberrations of the substrate and the longitudinal chromatic aberration of the eye.

As discussed before, as a variant, the one or more processors may be programmed to take into account a weighting factor Wf(λ) for determining the reference spectrum. The weighting factor Wf(λ) is determined based on the response curves of the three types of retinal cones: S, M and L (CIE data).

Such a weighting factor Wf(λ) may be derived based on response curves Response_S(λ), Response_M(λ), Response_L(λ) of the retinal cells detecting the light beam, for example on the basis of three types of cones noted in the following S, M and L.

The weighting factor Wf(λ) may be determined as a weighted average of the response of the three types of cones for each wavelength. It is for example calculated as Wf(λ)=ws*Response_S(λ)+wl*Response_L(λ)+wm*Response_M(λ), where ws, wm, and wl are three scalars to be defined to determine the weight of each cone response.

For example, the one or more processors 12 are programmed to determine the reference spectrum of the reference light beam by multiplying the initial spectrum of the light source Spect_source by the transmission or transmittance of the front part of the eye Trans_eye and by the weighting factor Wf:

$$Spect\_ref ( λ ) = Spect\_source {( λ )}^{*} Trans\_eye {( λ )}^{*} Wf ( λ ) .$$

The one or more processors 12 are then programmed to determine the test spectrum Spect_test taking into account the same weighting factor Wf, by multiplying the reference spectrum Spect_ref of the light source by the transmission or transmittance of the optical filter Trans_filter, or: Spect_test(λ)=Spect_ref(λ)*Trans_filter(λ). The weighting factor Wf is taken into account in the values of the reference spectrum Spect_ref.

The weighting factor Wf(λ) may be determined through the following process.

For each reference subject of a population of reference subjects and each filter of a group of reference filters, the ametropic shift is estimated through the method described above, without weighting factors. The shift in Equivalent Sphere of each eye of each subject is then measured for each filter.

Examples of implementation of these steps may be found in document US2020315448.

The values of the scalars ws, wm, and wl are then determined by optimization algorithms in order to minimize the difference between the ametropic shift estimated and the shift in Equivalent Sphere measured for each eye of each subject for each filter. In the present example, the optimization results are as follow:

$$ws = 0.05 , w m = - 0.5 , wl = 0. 87 .$$

The optimization space was not restricted here to positive factors.

The resulting weighting factor Wf(λ) is shown o FIG. 7. The weighting factor curve is positive everywhere.

Then, in order to test this method according to the first embodiment, the refraction shift model has been applied to a new set of 9 filters corresponding to commercial sunlenses and dopamine. A calculated shift was determined for each filter thanks to the method.

The spectra SA, SB, SC, SD, SE, SF, SG, SH, SI of each filter of the new set of 9 filters, noted sunlens or filter A to I, are shown on FIG. 8.

A measured value of the refraction shift introduced by each filter of this new set of filters was also measured for each reference subject of 30 reference subjects. The measured shift associated with each filter was obtained by averaging these measured values. The standard deviation of the 30 measured values was determined. The calculated shift determined with the refraction shift model was then compared with the measured shift. The results are summarized in the following table.

TABLE 1

| | | | Standard Deviation |
|---|---|---|---|
| | Measured | Calculated | for the measured |
| Filter | shift | shift | shift |
| Sunlens A | 0.146 | 0.077 | 0.102 |
| Sunlens B | −0.059 | 0.003 | 0.082 |
| Sunlens C | −0.043 | 0.034 | 0.095 |
| Sunlens D | 0.169 | 0.085 | 0.102 |
| Sunlens E | −0.022 | 0.027 | 0.067 |
| Sunlens F | 0.028 | 0.045 | 0.073 |
| Sunlens G | −0.029 | 0.022 | 0.077 |
| Sunlens H | 0.124 | 0.083 | 0.076 |
| Sunlens I | −0.204 | −0.218 | 0.112 |

These results are also summarized on FIG. 9, showing the measured and calculated shift values of each filter of the new set of 9 filters. The results show that over the 9 filters, there are 5 filters that induce a smaller refraction shift and 4 filters that induce a larger refraction shift.

More precisely, sunlenses B, C, E, F, G induce a refraction shift smaller than 0.05 D.

For these filters, the average measured refraction shift is smaller than the standard deviation across all 30 reference subjects (sigma −0.09 D). The average measured refraction shift is also small compared to the usual prescription tolerance in lens manufacturing, which is about 0.12 D. Finally, the average measured refraction shift is probably below the prescription sensitivity of most subjects. These refraction shifts are therefore considered to be negligible, and an accurate calculation of these shifts is not crucial.

Sunlenses A, D, H and I induce a refraction shift higher than 0.1 D.

For these filters, the average measured refraction shift is larger than the standard deviation of the measured shift values for the 30 reference subjects. Moreover, a fraction of subjects is sensitive to these values of refraction shift, meaning that they will be able to perceive it. These refraction shifts are therefore considered as significant.

For these filters, the refraction shift model described above correctly predicts the sign of the refraction shift. The average absolute error on the refraction shift is 0.05 D, which is satisfactory given the dispersion of the data.

The calculated and measured refraction shifts were here analyzed for a given light source having the spectrum shown on FIG. 3. This specific light source has higher radiance for determined wavelengths. To determine the influence of the light source on the calculated refraction shift, two different sunlight conditions were considered:
i) early sunrise, color temperature ˜3000 K
ii) sun at noon, color temperature ˜5500 K.

Other lighting conditions tested comprise a chart illuminated with a 7 LED light source referenced as ETDRS on FIG. 10 and the commercial screen having the spectrum of FIG. 3 noted CS.

The calculated refraction shift for both sunlight conditions, for the commercial screen CS having the spectrum of FIG. 3 and the chart illuminated with a 7 LED light source (EDTRS) were determined for a 45-year-old subject and compared.

The results are shown in FIG. 10. This FIG. 10 shows that, for the four filters A, D, H and I, the calculated refraction shift is relatively stable with respect to the light source. The calculated refraction shift may then be useful in different light conditions. The method according to the invention allows taking into account the spectral features of the light source used.

Therefore, the method described above allows determining with satisfactory accuracy the refraction shift introduced by a filter with different light sources without any measurement.

### Second Embodiment

According to the second embodiment, said one or more spectral features of the test light beam transmitted to the eye of the subject through the optical filter comprise a test spectrum of said test light beam on a predetermined global range of wavelengths.

This predetermined global range of wavelengths covers preferably the visible spectrum of light. It corresponds for example to the range of wavelengths comprised between 380 an 800 nm, or between 400 and 700 nm.

In this embodiment, this predetermined global range of wavelengths is divided into elementary range of wavelengths Bᵢ and an elementary shift value Sᵢ induced by each elementary range of wavelength Bᵢ is determined.

The elementary ranges of wavelengths Bᵢ correspond to bands of wavelengths, as schematically represented on FIG. 11.

The elementary ranges of wavelengths Bᵢ preferably cover the whole global range in a continuous manner with Bᵢ=[λᵢ,λ₂[=[λᵢ,λ₍ᵢ₊₁₎>[.

In practice, said one or more memories 11 have in memory said elementary shift values Sᵢ associated with predetermined elementary range of wavelengths Bᵢ, and said refraction shift model comprises a relationship between the refraction shift and a sum, for all of said elementary range of wavelengths Bᵢ comprised in said predetermined global range of wavelengths, of said elementary shift values Sᵢ weighted by an integral of the spectrum of said light transmitted to the eye through said filter OF within the corresponding elementary range of wavelengths Bᵢ.

As described in the first embodiment, for a given light source having a predetermined initial spectrum, the one or more processors 12 are programmed to determine a test spectrum of the test light beam. This test spectrum Spect_test(λ) is for example determined by multiplying the reference spectrum Spect_ref(λ) of the light source by the transmission or transmittance of the optical filter Trans_filter(λ), or: Spect_test(λ)=Spect_ref(λ)*Trans_filter(λ). The reference spectrum Spect_ref(λ) is for example determined by multiplying the initial spectrum of the light source Spect_source(λ) by the transmission or transmittance of the front part of the eye Trans_eye(λ), or: Spect_ref (λ)=Spect_source(λ)*Trans_eye(λ), as mentioned before.

The one or more processors could also be programmed to take into account a weighting factor such as the weighting factor described in the first embodiment, when determining the reference spectrum.

The calculated refraction shift takes into account the weighted sum on the global wavelength range of the elementary shift of each band of the global wavelength range, weighted by the integral of said test spectrum in that band.

Said refraction shift model provides a shift value equal to a ratio between said sum and an integral of said spectrum of said light transmitted to the eye through said filter within the predetermined global range of wavelengths.

The one or more processors 12 are then programmed to determine the calculated refraction shift with the following formula:

$$Calculated refraction shift = \frac{{Σ}_{i} Si {\int }_{λ i 1}^{λ i 2} Spect\_test ( λ ) d λ}{{\int }_{λ min}^{λ max} Spect\_test ( λ ) d λ}$$

Said elementary shift values Si are for example determined taking into account a database comprising a plurality of test spectra of light transmitted to an eye and the corresponding measured values of refraction shift in said eye, by minimizing the difference between each measured value of refraction shift and the value of refraction shift determined using said relationship and the test spectrum of light transmitted to the eye to which said measured value of refraction shift corresponds.

In practice, as an example of implementation of this second embodiment, it is possible to define six elementary wavelength ranges of 50 nm between 400 and 700 nm (as shown on FIG. 11).

The elementary shift values Sᵢ are optimized to minimize the difference between calculated refraction shift and measured refraction shift using a database of measured refraction shifts. As already mentioned in the example for the first embodiment, this database may be obtained by gathering refraction shifts measured using the 9 filters described as sunlenses A to I for 30 reference subjects.

The elementary shift values Sᵢ thus obtained are summarized in the following table 2.

TABLE 2

| Elementary range | Elementary shift value Si |
|---|---|
| 400 to 450 nm | −0.287 D |
| 450 to 500 nm | −0.078 D |
| 500 to 550 nm | −0.659 D |
| 550 to 600 nm | 0.011 D |
| 600 to 650 nm | 0.581 D |
| 650 to 700 nm | 0.159 D |

The comparison between the measured refraction shift and the calculated refraction shift for the 9 filters “sunlens A to I” is shown on FIG. 12.

We note that the calculated refraction shifts are quite close to the measured ones, especially on the four filters A, D, H and I The refraction shift model used in this second embodiment of the method is accurate.

In this example, the refraction shift model has 6 degrees of freedom, namely the 6 elementary refraction shift Sᵢ values, while the refraction shift model of the first embodiment only had 3 degrees of freedom, namely the 3 scalars of the weight factor.

In another example of the second embodiment, it is possible to define three elementary wavelength ranges of 100 nm between 400 and 700 nm.

The elementary shift values Sᵢ′ then determined are summarized in the following table 3.

TABLE 3

| Elementary range | Elementary shift value Si' |
|---|---|
| 400 to 500 nm | −0.160 D |
| 500 to 600 nm | −0.548 D |
| 600 to 700 nm | 0.580 D |

The results are almost as satisfactory with 3 elementary wavelength ranges as with 6 elementary wavelength ranges. The difference between the measured refraction shifts and the refraction shift calculated according to the method of the invention is less than 0.04 D for all filters.

Optionally, the refraction shift model can take into account the longitudinal chromatic aberration of the substrate of the optical filter, in addition to the longitudinal chromatic aberration of the eye. In that case, the elementary shift values Si need to be established for multiple substrate materials. When predicting the refraction shift for a given optical filter, the Si values corresponding to the specific substrate of the optical material should be used.

The device and methods of the invention therefore allow determining easily, quickly and without having to perform any measurement the refraction shift introduced by a filter OF for the eye E of a subject.

The refraction shift calculated thanks to the device and method described may be used for many different applications.

It may be used to modify the power of a corrective lens including a filter to compensate this refraction shift and ensure an accurate visual correction.

The power of the corrective lens may be fixed: it is then modified before manufacturing the eyewear. In this case, the device according to the invention can be embedded into the lens manufacturing calculator that designs a lens according to the chosen optical filter features.

It can also be modified in real time in the case of ophthalmic lenses having variable power and/or variable filter included. In this case, the power variation and/or the tint variation is adapted taking in consideration the refraction shift induced by the pending tint of the optical filter. The device according to the invention can be embedded in the frame wearing the ophthalmic lenses having variable power and/or variable filter or in any device adapted to communicate with a controller controlling of said ophthalmic lenses having variable power and/or variable filter, preferably wearable by the subject.

An adaptive eyewear for the subject according to the invention thus comprises:
ophthalmic lenses having variable power and/or a variable filter whose variations are controlled by a controller,
said device according to the invention and described above,
wherein said device is in communication with said controller and said controller is programmed to determine a power variation and/or a tint variation of said ophthalmic lenses taking into consideration the shift in the refraction of the eye of the subject determined by said device.

Further, the refraction shift model could be modified to take into account the index of the lens substrate and/or of the material of the filter.

1. A device for determining a shift in a refraction value of an eye of a subject lighted by a light beam emitted by a light source, said shift being induced by an optical filter through which said light beam is transmitted to the eye of the subject, said device comprising:
one or more memories, and
one or more processors, wherein:
said one or more memories store:
one or more values of one or more initial spectral features of said light source,
one or more values of one or more optical features of said optical filter, and
a refraction shift model linking a magnitude associated with said shift and one or more spectral features of the light beam transmitted to the eye of the subject; and wherein
said one or more processors are programmed to perform:
determining one or more values of one or more spectral features of said light beam transmitted to the eye of the subject through said optical filter based on said one or more values of one or more initial spectral features of said light source and said one or more values of one or more optical features of said optical filter; and
determining said shift in the refraction of the eye of the subject based on said refraction shift model and said values of the spectral features of said light beam transmitted to the eye of the subject through the optical filter.

2. The device according to claim 1, wherein said one or more values of one or more initial spectral features of said light source comprise an initial spectrum of said light emitted by said light source and said one or more values of one or more optical features of said optical filter comprise values quantifying the transmission of light through said optical filter.

3. The device according to claim 1, wherein said one or more processors are programmed to determine a spectrum of said light beam transmitted to the eye of the subject through said optical filter for determining said one or more values of one or more spectral features of said light beam transmitted to the eye of the subject though said optical filter.

4. The device according to claim 1, wherein said one or more memories further store data relative to one or more optical features of the eye of the subject and said one or more processors are programmed to take into account these data for determining one or more values of one or more spectral features of said light beam transmitted to the eye of the subject through said optical filter.

5. The device according to claim 4, wherein said one or more memories store weighting factors representative of a light sensitivity of the retina of the eye of the subject at one or more wavelengths and wherein said one or more processors are programmed to determine said one or more values of one or more spectral features of said light beam transmitted to the eye of the subject through said optical filter taking into account these weighting factors.

6. The device according to claim 1, wherein said one or more spectral features of the light beam transmitted to the eye of the subject through said optical filter comprise a statistical value of wavelength representative of the wavelength distribution of a spectrum of said light beam transmitted to the eye of the subject through said optical filter.

7. The device according to claim 6, wherein said statistical value of wavelength representative of the wavelength distribution of said spectrum comprises a wavelength barycenter of said spectrum.

8. The device according to claim 6, wherein said one or more memories store data relative to a longitudinal chromatic aberration of the eye depending on a wavelength of a light beam transmitted to the eye and wherein said refraction shift model comprises a relationship between the shift in refraction of the eye of the subject and the longitudinal chromatic aberration of the eye of the subject at said statistical value of wavelength.

9. The device according to claim 8, wherein:
said one or more values of one or more initial spectral features of said light source comprise an initial spectrum of said light emitted by said light source;
said one or more processors are programmed to determine a reference statistical value of wavelength representative of the wavelength distribution of a reference spectrum of a reference light beam emitted by the light source and transmitted to the eye of the subject in the absence of the optical filter based on said initial spectrum of said light emitted by said light source; and
said refraction shift model takes into account the longitudinal chromatic aberration of the eye of the subject at said reference statistical value of wavelength.

10. The device according to claim 9, wherein said refraction shift model provides a shift value equal to the difference between longitudinal chromatic aberration of the eye at the statistical value of wavelength and the longitudinal chromatic aberration of the eye at said reference statistical value of wavelength.

11. The device according to claim 1, wherein said one or more spectral features of the light beam transmitted to the eye of the subject through the optical filter comprise a spectrum of said light beam transmitted to the eye through said optical filter on a predetermined global range of wavelengths.

12. The device according to claim 11, wherein said one or more memories store elementary shift values associated with predetermined elementary range of wavelengths, and said refraction shift model comprises a relationship between the refraction shift and a sum, for all of said elementary range of wavelengths comprised in said predetermined global range of wavelengths, of said elementary shift values weighted by an integral of the spectrum of said light transmitted to the eye through said filter within the corresponding elementary range of wavelengths.

13. The device according to claim 12, wherein said refraction shift model provides a shift value equal to a ratio between said sum and an integral of said spectrum of said light transmitted to the eye through said optical filter within the predetermined global range of wavelengths.

14. The device according to claim 12, wherein said elementary shift values are determined taking into account a database comprising a plurality of test spectra of light transmitted to an eye and the corresponding measured values of refraction shift in said eye, by minimizing the difference between each measured value of refraction shift and a calculated value of refraction shift, said calculated value of the refraction shift being determined using said relationship and the test spectrum of light transmitted to the eye to which said measured value of refraction shift corresponds.

15. Adaptive eyewear for a subject comprising:
ophthalmic lenses having variable power and/or a variable filter whose variations are controlled by a controller, and
the device according to claim 1,
wherein said device is in communication with said controller and said controller is programmed to determine a power variation and/or a tint variation of said ophthalmic lenses taking into consideration the shift in the refraction of the eye of the subject determined by said device.

16. A method for determining a shift in a refraction value of an eye of a subject lighted by a light beam emitted by a light source, said shift being induced by an optical filter through which said light beam is transmitted to the eye of the subject, said device comprising one or more memories and one or more processors, the method comprising:
determining one or more values of one or more initial spectral features of said light source,
determining one or more values of one or more optical features of said optical filter,
providing a refraction shift model linking a magnitude associated with said shift and one or more spectral features of the light beam transmitted to the eye of the subject;
determining one or more values of one or more spectral features of said light beam transmitted to the eye of the subject through said optical filter based on said one or more values of one or more initial spectral features of said light source and said one or more values of one or more optical features of said optical filter; and
determining said shift in the refraction of the eye of the subject based on said refraction shift model and said values of the spectral features of said light beam transmitted to the eye of the subject through the optical filter.
